(19) United States
(12) Patent Application Publication
(10) Pub. No.: US 2024/0112595 A1
(43) Pub. Date: Apr. 4, 2024

(54) Eye Drop Tracker

(71) Applicant: Carolynn Reppert Sears, Pound Ridge, NY (US)

(72) Inventor: Carolynn Reppert Sears, Pound Ridge, NY (US)

(21) Appl. No.: 17/952,266

(22) Filed: Sep. 25, 2022

(51) Int. Cl. G09B 19/00; A61B 50/33; A61F 9/00

(52) U.S. Cl. CPC G09B 19/00; A61B 50/33; A61F 9/0008

(57) ABSTRACT

An aspect to the device is an arrangement of pegs and holes for general time indication serving as visual and mechanical reminders through passive means for multiple medicament distribution. The device and method contain, store, organize, and dispense multiple different medications per eye for systematic instillation and tracking throughout the day. As a container, the device prevents a potential mix-up with other squeeze bottles. The economical, easy-to-use device and method support in-office instruction, are adaptable to different regimes, portable, and may be used in conjunction with other aids, e.g. eye drop dispenser, alarms, logs and Apps. At end-of-life, the product can be recycled or composted. Thus, the reader will see the present embodiment provides support for patients, making it easier to follow doctor's orders and establish a home-care routine to prevent infection, minimize swelling, reduce irritation, sooth, and help eyes to heal fully.

### BACKGROUND OF THE INVENTION

### Technical Field

The present embodiment relates to medicament distribution means, particularly for multiple ophthalmologic liquids, also known as eye drops, in squeeze dropper bottles.

### Problem to be Solved

Patent adherence to eyedrop regimes is a well-known problem. To grasp the scope of the problem, consider that over two million cataract removal surgeries are performed annually in the U.S. and patience noncompliance is estimated to be at 40% and possibly as high as 80%. Forgetting to instill eye drops leads to negative health impacts, more doctor visits, medications or surgeries. Whether short-term or long-term, simple or complex daily regimes can be challenging to remember and track.

### PRIOR ART

In this section the advantages and disadvantages of prior art that appears to be relevant are considered in three areas: in regards to individual patients, as an in-office instructional device, and impacts on the environment.

First, patient needs differ for short-term or long-term support regarding the daily instillation of multiple eyedrops in different eyes, following different regimes. Some prior art is customized for the patient (Mogilefsky, Holmberg). While beneficial, the extra cost may be prohibitive to many patients. Another form of prior art references or includes electronics, processor circuits (Taylor), or soft-ware (Barsky). These forms of prior art may also require of patients an additional expense plus familiarity with technology. Another form of prior art attaches to individual medicine bottles (Kramer; Dossin) and may be difficult for some patients to manipulate. In addition, an attachment does not necessarily assist the patient with following a complex regime or promote a routine. Other prior art is missing a reminder system (Askew; [symbol] ) or offers weak reminder systems, e.g. a write-on surface (Horn), inscriptions on a board (Postow), charts (Nellhaus), a sheet with color-coded fields (Elppert), and adhesive graphics (Barsky, cited previously). These generally do not address the complexity of multiple eye drops being instilled to two different eyes on two different regimes and lack a reminder or tracking system in regards to the actual utilization of eye drops at the prescribed interval. At the same time, each one of these forms of prior art may serve some individuals as the reasons for noncompliance are rooted in physical, cognitive, or behavioral issues which vary from patient to patient. Hence a variety of solutions is needed to address individual differences. The need for other solutions is great.

Second, medical staff struggle to clearly describe a prescribed regime to patients and improve patient compliance. None of the aforementioned prior art present as useful in-office instructional devices with manipulative features to demonstrate the prescribed regime to patients.

Third, none of the aforementioned prior art address the hidden costs and impacts of manufactured goods on the environment and society. Prior art which reference a processor circuit (Taylor, cited previously) would increase the use of batteries. Toxic metals in batteries are known carcinogens and improperly disposed batteries contribute to water and air pollution. Prior art made of plastic introduces a material into the environment which affects the climate at every stage of its life cycle. Historically speaking, prior art does not describe the materials used in construction and the disposal at the end of the product's use.

To summarize, no single device or method can adequately address the multiple reasons for patient noncompliance to the short-term or long-term daily instillation of one or more eyedrops in different eyes, following different regimes. A patent and market search, to the best of my knowledge, show no single device or method addressing the problem by providing a simple, easy-to-use, inexpensive device to provide a reminder and tracking system for at-home patient care, support in-office patient-doctor communication, and address some of the costs to society associated with production and end-of-life product disposal.

### BRIEF SUMMARY OF THE INVENTION

The present embodiment offers another option to address a long-standing need: an inexpensive device combined with an easy-to-follow method to simplify eye care for thousands of people, the basis of which is the novel use of pegs and holes. While the following description and drawings seem obvious, the patent search confirms a curiosity: the failure of others to conceptualize such a simple, yet elegant device sooner. In the office, the present embodiment is a manipulative device for doctors and medical staff to use while explaining complex instructions to patients. At home, it provides visual reminders and means for the patient to track his or her compliance to a prescribed regime. In addition, the present embodiment is constructed of recycled or sustainable materials that can be recycled or composted when no longer needed.

A first embodiment includes a device and method for in-office instruction and at-home use. A plurality of prescribed medications in eye drop squeeze bottles are to be contained within said device. Said device and method use pegs and holes to passively remind a human regarding the daily instillation of prescribed eye drops and aid tracking daily instillation of said medications. Additional embodiments for said device are methods for adhering to different prescribed regimes.

### BRIEF DESCRIPTION OF THE DRAWING

FIG. 1 is perspective of the exterior and interior of the device.

FIG. 2 illustrates a component part of the device, a peg.

### REFERENCE NUMERALS

110 Tray with lid
120 Plinth
130 Holes forming two parallel columns
140A Left Storage Chamber
140B Right Storage Chamber

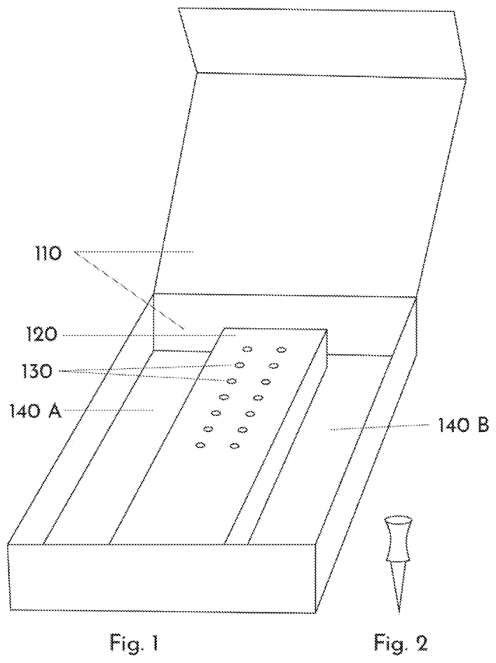

### Detailed Description of the Invention—FIGS. 1-2

The present embodiment provides a simple, inexpensive, nontechnical solution to the problem. The device to be described utilizes pegs and holes to provide a manipulative for demonstrating the prescribed regime in the medical office and a visual reminder and tracking system for home use regarding the actual instillation of eye drops at prescribed intervals throughout the day. Said device stores, organizes and dispenses multiple medications per eye for systematic instillation and tracking; it is easy to use and manipulate by the patient. Said device can prevent possible mix-ups with other medications, is portable, and may be used in conjunction with other aids, e.g. eye drop dispenser, alarms, logs and applications, downloaded to a mobile device, known as Apps. At end-of-life, the device and component parts can be recycled or composted. These and other advantages for one or more aspects will become more apparent from a consideration of the ensuing description of said device:

A rectangular container comprising lower and upper members which close or open relative to each other to form a covered container similar to a pizza box, said upper member forming a lid and said lower member forming a tray. The lower member, the tray, containing a rectangular plinth centered lengthwise and delineating the interior space, one-third to the left and one-third to the right. On the surface of the plinth are two parallel columns comprised of holes. Included within the container are a plurality of component parts, referred to as pegs, all of the same predetermined shape and size so as to snuggly fit said holes.

A device and method create a system based on the novel use of pegs and holes that is useful to thousands of patients typically under care for treatments such as post-operative cataract removal surgery, Lasik surgery, glaucoma, thyroid eye disease, also known as Grave's disease, or artificial tears. The problem to be solved is the challenge of patient adherence to either a simple or complex drop schedule involving one or both eyes, one or more medications, with changes to the regime, and over a period of time. The device and method support in-office communication between patient and medical staff around the prescribed regime. The device and method support out-patient home care and provide a physical reminder and memory aid making it easier for care-givers and patients to follow and track prescribed home care regimes in order to take care of their eyes.

### Operation FIGS. 1-2

The device represented in FIGS. 1-2 contain, store, organize, and dispense up to five different medications per eye for systematic instillation and with pegs and holes provide visual reminder and tracking throughout the day.

FIG. 1 110 is a perspective of the exterior and interior of the device showing lower and upper members which close or open relative to each other to form a covered container, said upper member forming a lid and said lower member forming a tray. When closed the device is portable. The lower member, the tray, containing a rectangular plinth 120 centered and delineating the interior space lengthwise such that one or more 5 ml dropper bottles can be stored and held separately for each eye. This aspect functions to prevent a potential mix-up of squeeze bottles.

FIG. 1 details the interior of the tray 110 showing a plinth 120 centered and delineating the interior space lengthwise. A storage chamber 140A to the left functions to contain medications for the left eye and a storage chamber 140B to the right functions to contain medications for the right eye. On the plinth 120 are two parallel columns of holes 130 that function to hold pegs, a component part of the device and system illustrated in FIG. 2.

The function of said pegs FIG. 2 and the placement of said pegs is to remind the user and track the instillation of eye drops. Each eye may be on the same or on a different regime. At the start of the day, peg(s) inserted in the left column on the plinth 220 and in the right column function to indicate the number of times to instill medications in each eye on that day. For example, two pegs in two holes in the left column function to represent instilling drops twice that day for the left eye while four pegs in four holes in the right column function to represent instilling drops 4 times that day for the right eye. After instilling eye drops as prescribed, a peg is removed and placed in the storage chamber 140A or 140B for the remainder of the day. The function of the placement of the pegs is to visually remind and track user's compliance with the prescribed regime.

By way of example, the preceding description of the operation and function of the device and component parts presents a portion of one commonly prescribed regime. Thus, the reader will see that this simple, economical and environmentally-friendly device and method supports better care of the eyes. Other variations are possible with several outlined in the appended claims.

I claim:

1. A device and method of usage for demonstrating the prescribed regime in the medical office and reminding a human and tracking the daily at-home instillation of a plurality of prescribed medications in eye drop squeeze bottles contained therein.

2. The device comprising a-e:
a. a container, rectangular of predetermined dimensions, comprising lower and upper members which close or open relative to each other to form a covered container, said lower member forming a tray and said upper member forming a lid so as to contain a plurality of eye drop squeeze bottles, hereafter referred to as squeeze bottles.
b. a plinth, rectangular of predetermined dimensions, centered in the tray and delineating the interior space lengthwise, one-third to the left and one-third to the right, so as to separate a plurality of squeeze bottles.
c. said plinth with two parallel rows of holes on the surface so as to hold pegs.
d. a plurality of said pegs of predetermined size so as to snuggly fit said holes.
e. an instruction manual with charts to explain method of use.

3. The device of claim 1 wherein holds squeeze bottles separately for the left and right eye and from other similar squeeze bottles with contents not to be instilled in the eye of human, e.g. ear drops, medication for a pet.

4. The device of claim 1 is compatible with commonly used eye drop squeeze bottles.

5. The device of claim 1 wherein materials are from recycled or renewable sources and at end of life are recyclable and/or compostable.

6. The device of claim 1 wherein materials used reduce the cost and weight without compromising durability and utility.

7. The method of device usage of claim 1 being a visual reminder and tracking of the daily instillation of multiple prescribed medications with means comprising a-h:
a. Storing prescribed medications in squeeze bottles in said device, typically for the left eye on the left side of said plinth and for the right eye on the right side of said plinth.
b. Utilizing instructions and chart per Doctor's prescription and in-office demonstration.
c. Placing said pegs into the holes in the columns on said plinth, corresponding to the prescribed number of times to instill drops daily for each eye, typically for the left eye in the holes of the left column and for the right eye in the holes of the right column of said plinth.
d. Isolating temporarily said squeeze bottle(s) in use for one eye from other bottles in the tray and returning each of the bottle(s) one-by-one after use to the storage area of the device.
e. Removing one said peg and laying it in the storage area after instilling medications as prescribed for that eye.
f. Repeating d. and e. for the second eye.
g. Remaining said pegs in holes providing a visual reminder and tracking of adherence to the daily schedule.
h. Adjusting the daily schedule and routine over time as prescribed with annotations on said chart and adjustments to pegs used and bottles stored in said device.

8. The method of claim 7 wherein is adaptable to other prescribed regimes, known hereafter as embodiments.

9. Directions of four said embodiments are summarized and outlined here as examples wherein said device and method are not limited to the following embodiments a-d:
a. Example #1 for post-cataract surgery
Week 1: 4 times/day, instill a drop of all three medications
At the end of the week discontinue using the antibiotic (beige cap)
Week 2: 3 times/day, instill a drop of both anti-inflammatory medications
Week 3: 2 times/day, instill a drop of both anti-inflammatory medications
Week 4: 1 time/day, instill a drop of both anti-inflammatory medications
At the start of the week, circle the #times/day to instill drops on the chart below and insert the pegs into the columns representing the left and right eye as prescribed.

| EYE | L | EYE | R |
|---|---|---|---|
| Wk 1 | 4 | Wk 1 | 4 |
| Wk 2 | 3 | Wk 2 | 3 |
| Wk 3 | 2 | Wk 3 | 2 |
| Wk 4 | 1 | Wk 4 | 1 |

b. Example #2. For medications(s) to be instilled twice a day
Insert two pegs into two holes in each column representing the left and right eye as prescribed.

| | Eye | L | R |
|---|---|---|---|
| Week 1 | #times/day | 2 | 2 |
| Week 2 | | 2 | 2 |

c. Example #3. For one medication used 2× daily, e.g. Glaucoma Patients
At the beginning of the week, insert pegs into the holes of both columns, representing morning and night, for twice-a-day application as prescribed.

| | |
|---|---|
| am | pm |

SUN
MON
TUES
WED
TH
FRI
SAT
d. Example #4 Using two trays for a dosage schedule like this:
Moxifloxacin (beige cap) 2×/day for 1 wk
Difluprednate (white cap) 2×/day for 2 wks
1×/day at night for 2 wks
Ilevro (grey cap) 1×/day at night for 6 wks
Use two trays (one for the left eye and the other for the right): Place dropper bottles for Moxifloxacin and Difluprenate in the storage space on the left. Add pegs to the left-hand column as reminders. Use the storage space on the right and the right-hand column for tracking Ilevro.

| Left Eye | #times/day | Moxi. & Diflu | Ilevro |
|---|---|---|---|
| Week 1 | | 2 | 1 |
| Remove | | | |
| Moxifloxzcin | | Diflu | Ilevro |
| Week 2 | | 2 | 1 |
| Week 3 | | 1 | 1 |
| Week 4 | | 1 | 1 |
| Week 5 | | | 1 |
| Week 6 | | | 1 |

Set up tray #2 for the right eye in the same way:

| Right Eye | #times/day | Moxi. & Diflu | Ilevro |
|---|---|---|---|
| Week 1 | | 2 | 1 |
| Remove | | | |
| Moxifloxzcin | | Diflu | Ilevro |
| Week 2 | | 2 | 1 |
| Week 3 | | 1 | 1 |
| Week 4 | | 1 | 1 |
| Week 5 | | | 1 |
| Week 6 | | | 1 |

10. Said device and method of usage of claim 1 may be used independently or in conjunction with other devices e.g. eye drop bottle squeezer, logs, alarms, and Apps.
The device and method of usage of claim 1 whereby through the unique use of pegs and holes a human is instructed, visually reminded, and able to track the instillation of multiple prescribed eye drops stored, organized and dispensed within.
